(19) United States
(12) Patent Application Publication
(10) Pub. No.: US 2004/0193452 A1
(43) Pub. Date: Sep. 30, 2004

(54) Method and system for computerized sexual function assessment of female users

(76) Inventor: Laura Berman, Chicago, IL (US)

Correspondence Address: MANATT PHELPS AND PHILLIPS, ROBERT D. BECKER, 1001 PAGE MILL ROAD, BUILDING 2, PALO ALTO, CA, 94304, US

(21) Appl. No.: 10/752,771

(22) Filed: Jan. 6, 2004

Related U.S. Application Data
(60) Provisional application No. 60/438,379, filed on Jan. 6, 2003.

(51) Int. Cl. G06F 17/60; A61B 10/00; A61B 5/00

(52) U.S. Cl. 705/2; 128/920; 600/300

(57) ABSTRACT

The present invention is directed to a method and system for performing an automated sexual function assessment of a female user having a link between the user's computer and a central computer. After the user registers, obtains a password and a screen name, and provides the user's demographic information to a database of the central computer, a number of female sexual dysfunction (FSD) questions are displayed on the user's computer screen in the first phase. Phases two and three questions are then displayed so long as the responses to such FSD questions, that are sent to a user file in the database, indicate a possibility the user has some degree of FSD. The second phase questions relate to psychological causal factors for FSD. The third phase questions relate to medical factors impacting on sexual function. An additional set of questions relating to the quality of the user's orgasms are usually displayed during the course of the assessment. At the end of the assessment, an evaluation is made of the user's responses and a determination is made whether the user is a suitable candidate for medical treatment to enhance sexual function if there are no psychological casual or medical factors for FSD and user has no more than a fair satisfaction level on the question of orgasm enjoyment based on an evaluation of the quality of the user's orgasms.

This application claims the benefit of prior U.S. provisional application Ser. No. 60/438,379 filed on Jan. 6, 2003.

### FIELD OF THE INVENTION

The present invention relates to a method and system for assessing the sexual function of female users to provide a preliminary diagnosis of female sexual dysfunction (FSD).

### BACKGROUND OF THE INVENTION

A large number of females suffer from FSD without knowing it. Even if they are aware they have a problem, they often do not seek any form of counseling or medical or psychological therapy because of cost, time constraints, inconvenience, or embarrassment. The early detection and treatment of FSD could result in a healthier more fulfilling life for millions of women.

Sexual dysfunction is highly prevalent, affecting 43% of the women under sixty in the United States that is significantly higher than men, who suffer at a rate of 31%; based on research done by Laumann, et. al., “Sexual Dysfunction in the United States: Prevalence and Predictors, Journal of the American Medical Association, v. 281, pp. 537-544, 1999; also see Berman, L. A., and Berman, J. R., For Women Only—A Revolutionary Guide to Reclaiming Your Sex Life, Henry Holt and Company, 2001, the contents of which are incorporated herein by reference. It is obvious that in the United States alone there are a large number of women that have access to personal computers, who would benefit greatly if they had access to a computerized system of sexual function assessment. Also, there is a strong need to identify and/or to rule out female sexual dysfunction in order to further medical and pharmacological research, without requiring expensive and sometimes embarrassing visits to clinical therapists and other health professionals, who may not have specific training in FSD diagnosis.

Research reports that 28% of girls in the United States have been subjected to some form of childhood sexual abuse (CSA) or otherwise sexually exploited before the age of 14. See Vanderkolk, B. A., (1987), Psychological Trauma; Washington D.C.: American Psychiatric Press. Studies have indicated that at least half as many women with as without unresolved CSA histories responded positively to sildenafil (Viagra). Female survivors of unresolved CSA who are suffering with related FSD may not obtain a satisfactory sexual response with sildenafil and should seek effective psychosexual treatment. See Berman, L. A.; Berman, J. R.; Bruck, D.; Pawar, R.; Goldstein, I, (2001), Pharmacotherapy or Psychotherapy?: Effective Treatment for FSD Related to Unresolved Childhood Sexual Abuse, Journal of Sex and Marital Therapy, vol. 27, no. 5, pp. 421-425, 2001, Brunner-Routledge, the contents of which are incorporated herein by reference.

During a period from 1992-1999, women comprised a growing proportion of persons living with HIV/AIDS, one of the most devastating medical factors impacting sexual function. In 1992, women accounted for 14% of adults/adolescents living with HIV/AIDS. By 1999 the proportion had grown to 20%. There has been more than triple the number of women with HIV infections from 1985 to 1999. There is a need for a low cost and completely private FSD assessment of women that could result in at least a preliminary diagnosis and a recommendation for immediate medical treatment for HIV.

Numerous pharmaceutical companies have taken on the task of finding an effective treatment for FSD, but one of the most significant challenges has been the complex nature of female sexuality. When research was done on the effectiveness of Viagra in males, it was found that only 10% of male erectile dysfunction is psychogenic and in that group, Viagra resolved psychogenic erectile dysfunction over 80% of the time. The research in women has demonstrated that their sexual response is much more complex and if they are not feeling good enough about themselves, their bodies and the person they are with, medication does help. In these cases, even when there are medical causal factors, psychotherapy must be carried out first or in conjunction with medical treatment in order for it to be effective. The challenge for the pharmaceutical industry has been in identifying those women who may indeed benefit from medication and are not better suited to therapy. There is a need for an instrument to serve as a useful tool for identifying these women and helping healthcare providers make the most effective treatment decisions for their patients.

There is also a pressing need for a method and system of gathering information on individual females that can provide a low cost, private, simple and very efficient preliminary diagnosis of FSD and to identify any medical or psychological factors that may impact on sexual function.

### SUMMARY OF THE INVENTION

The present invention solves the problem of providing a simple, reliable, secure online method and system for assessment of FSD. The method of the present invention is referred to herein as the Female Sexual Dysfunction Assessment or simply the FSDA method.

A first phase of the FSDA method sequentially displays, on the screen of the user's computer system, a number of questions to determine a preliminary sexual dysfunction diagnosis. The assessment is completed if all of the user's responses are never and there is no need for the user to go on to a second phase of the method.

In a second phase, a number of questions are displayed either on the user's monitor screen or one to which the user has access in order to determine if the user has any psychological red flags or casual factors for FSD. The answers to these questions that are sent to the database for evaluation are much more complex than in the first phase. The user continues to a third phase regardless of the answers to questions in the second phase.

A third phase of the method covers questions that are all related to the state of the woman's health and are within the broad definition of medical factors for FSD.

In a preferred embodiment of the FSDA method, the medical factors questions fall into one or more the following categories: tobacco use, alcohol use, medical history, current medications, menstrual status, and pain problems. It is preferred that during the third phase, the user at least answers questions relating to medical history, current medications, menstrual status, and pain problems.

After all of the questions, answers and other responses to the first, second and third phases have been sent to a database within a central computer system, the FSDA method enters into the evaluation phase. A determination is made whether the user is a suitable candidate for psychological treatment or the use of pharmacological options to enhance sexual function or a combination of both treatments. It is critical to a women's health that pharmacological options for FSD not be recommended if the user requires psychological treatment for sexual enhancement or medical therapy for medical problems other than for sexual enhancement.

The system of the present invention is referred to herein as the FSDA system for performing the FSDA method and uses a link between the user's computer system and the central computer system. Briefly the systems includes:

(1) a modem or other remote communication system for initiating a remote communication from the user's computer system with the central computer system;

(2) first phase storage on a hard drive within the central computer system for storing the first phase questions of the FSDA;

(3) second phase storage for storing the second phase questions of the FSDA depending on the responses received in the first phase;

(4) third phase storage for storing the second phase questions of the FSDA depending on the responses received in the second phase;

(5) display screen on the user's computer system for displaying the plurality of questions received via the remote communication system;

(6) response storage for storing the responses to the plurality of questions in a separate user file; and

(7) an instrument programmed into the central computer system for evaluating the responses from the plurality of questions during the first, second, and third phases if any of the responses of the first phase questions are other than never and determining whether the user is a suitable candidate for medical therapy or psychological therapy to enhance sexual function or both of these therapies.

### BRIEF DESCRIPTION OF THE DRAWINGS

The foregoing aspects and the attendant advantages of the present invention will become more readily appreciated by reference to the following detailed description, when taken in conjunction with the accompanying drawings, wherein:

FIG. 1 is a block diagram showing a typical system for a user to communicate through a conventional communication protocol to a central computer system of the method of the present invention;

FIG. 2 is a flow chart of a preferred embodiment of the method of the present invention administered by the computer system of FIG. 1;

FIG. 3 is a flow chart showing various algorithmic paths for users suffering from hypoactive sexual desire disorder used in one of the steps of the preferred embodiment of the method of the present invention;

FIG. 4 is a flow chart showing various algorithmic paths for users suffering from sexual arousal disorder used in one of the steps of the preferred embodiment of the method of the present invention;

FIG. 5 is a flow chart showing various algorithmic paths for users suffering from orgasmic disorder used in one of the steps of the preferred embodiment of the method of the present invention; and

FIG. 6 is a flow chart showing various algorithmic paths for users suffering from sexual pain disorders used in one of the steps of the preferred embodiment of the method of the present invention.

### DETAILED DESCRIPTION OF THE INVENTION

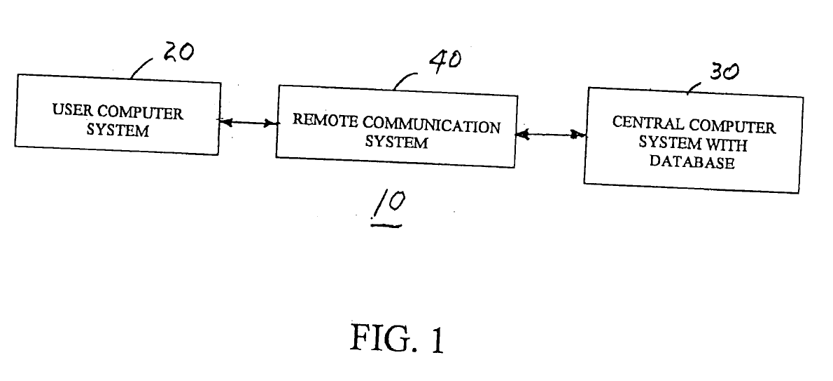

Referring to FIG. 1, a typical system 10 that a user employs to carry out the method of the present invention is shown. A standalone user computer system 20 is operably connected to a controlling central computer system 30 through modem 30 or any other suitable remote communication system. User computer system 20 is a client computer system that is typically at the user's home. However, alternate remote sites can be made available for any user wishing to be a participant in the FSDA. The controlling computer system 30 is programmed to carryout the FSDA method. The portion of the program that carries out the step of evaluating the responses and making recommendations or providing a preliminary diagnosis to the user is referred herein as the FSDA instrument. Specifically, computer system 30 contains sufficient storage space on a hard drive or a plurality of hard drives for storage of the programs for running all of the steps of the FSDA method, for containing the information received from the users in separate files, and for evaluating the information received from the users. Computer system 30 is generally remotely located at an office (not shown). It is not critical that user system 30 and central computer system 30 be the same type of system and any system known by those skilled in the art such as Macintosh, PC, UNIX system, VAX, and others can be used.

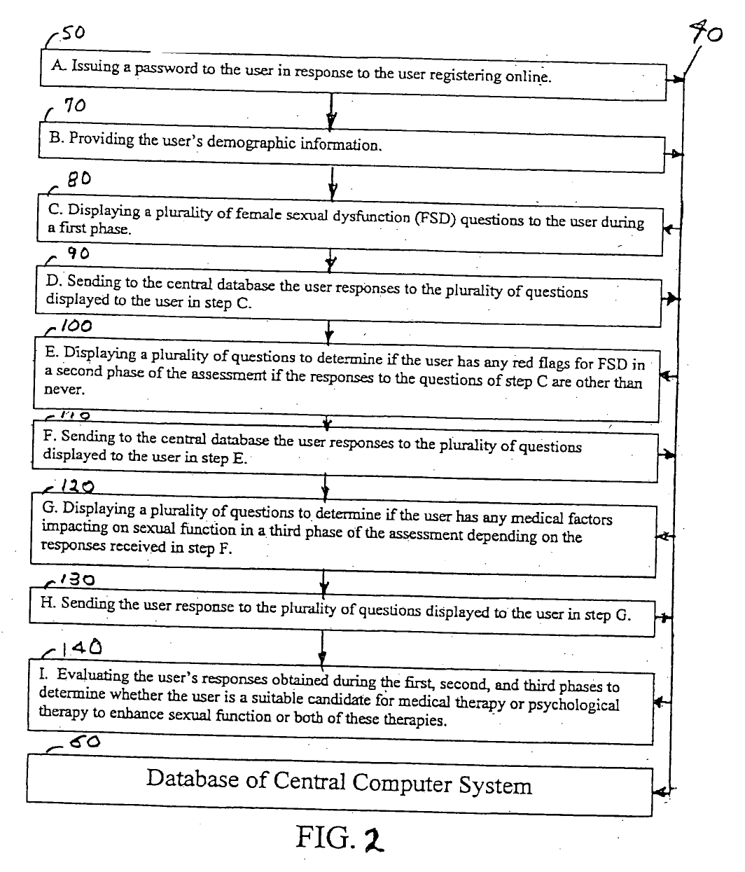

FIG. 2 briefly summarizes a preferred embodiment of the method of the present invention where step 50 initiates the method with the user registering through the communication protocol 40 to database 60 of central computer system 30. The FSDA method has all participants register to select a password and preferably to select a screen name to further maintain anonymity. The central computer system 30 generates the web pages for the registration, including a disclaimer agreement relating to taking the FSDA evaluation on a preliminary basis only, and maintains password and screen name control, database storage of all responses of the users, feedback capacity from users, administration pages, instant capacity for users to change font and colors, and similar formatting changes. High speed/high capacity database storage is integrated into the program with nothing being stored separately or on a different server. There is no need, nor is there any use, for cookies, i.e., a message given to a Web browser by a Web server where the browser stores the message in a text file and the message is then sent back to the server each time the browser requests a page from the server.

The FSDA method is administered and scored, with results then displayed to the user and stored entirely on the World Wide Web with password protection. Users can download or print their reports upon completing the FSDA method. Two 4 to 5 page reports are generated; one for the user that takes part in the method and one for professionals that may be called in as advisors for the FSDA method, such as physicians, sex therapists, and similar specialists. The professional version of the report can be accessed only via a password assigned to pre-approved professionals.

The FSDA method examines approximately twenty general areas relevant to a women's sexual health; in increasing depth depending on detection of problems at the initial screening level. Healthy women (i.e., those that do not suffer from FSD), complete the FSDA quickly, with a minimum of exposure to more in depth questions.

Step 70 of the method shown in FIG. 2 is for the user to provide certain demographic information that is useful in establishing a proper diagnosis. The demographic information includes the user's address, age, racial background, education, employment status, and household income. After the demographic information has been stored in the database, the assessment begins with a series of World Wide Web pages using a conventional Web browser that are displayed on the screen of a monitor of the user's computer system 20 or one to which the user has access. (See pages 2-3 of the Appendix for the list of the type of demographic information that is requested and a summary of possible responses.)

The users have total anonymity with no direct connection to the central database 60 on their computer systems 20. No Java is required or used on the user's computer system 20, which acts differently on differing computers. Everything is server managed and designed to speed loading pages on the user's computer 20; e.g., no PICT supported by graphics programs that run on Macintosh computers, jpg, or other picture formats are used anywhere in the FSDA method as they could slow loading pages; all entries are in HTML (HyperText Markup Language) generated on the user's computer 20. The FSDA method presents a totally interactive experience with the user. It allows a user to interact with the FSDA instrument for a short period of time and to obtain the advice that is built into the FSDA program. The advice is personalized based on the user's personal life situation and other information supplied by the user during the FSDA method.

PHASE ONE: In step 80 shown on FIG. 2, questions according to the National Consensus Panel of the American Foundation of Urologic Disease (AFUD) to determine a sexual dysfunction diagnosis are sequentially displayed on the user's screen. The questions of Phase One fall into one or more of the following areas that traditionally are considered sexual dysfunction problems: (1) hypoactive desire disorder, (2) sexual arousal disorder, (3) orgasmic disorder, (4) sexual aversion disorder, and (5) sexual pain disorders. Preferably all of the foregoing areas are included in the Phase One.

In step 90, the answers to the foregoing five questions described below of Phase One are sent via communication system 40 to database 60 and will determine whether the user is likely to be suffering from sexual dysfunction problems. It is preferred that all of these Phase One questions be truthfully answered by each user before the FSDA method permits the user to move to Phases Two and Three.

The first Phase One question relates to hypoactive desire disorder, which include a lack of interest in having sex or a lessening in the desire to have sex. A typical hypoactive desire disorder question that is displayed on the monitor screen is: Do you ever lack sexual interest or sexual thoughts, fantasies or motivation? The user then hits the icon or otherwise responds to the question with a “never,” “occasionally,” “often” or “always.”

If the user responds with an answer of “often,” the screen displays an interim report that can be printed and the FSDA instrument will report that the user has indicated a significant lack of sexual interest or sexual thoughts, fantasies, or motivation. On the other hand, if the user responds with an “always,” the screen displays an interim report that the user's lack of sexual interest or sexual thoughts, fantasies, or motivation is considered to be a sign of sexual dysfunction.

If any one of the Phase One questions discussed in this section is answered with “never” or “occasionally,” no interim report will result with respect to that question and the user will be prompted to the continue to the next question.

If any of the Phase One questions discussed in this section is answered with “often” or “always,” a series of additional questions will be displayed on the screen. These questions will require the user to use a keyboard, mouse or other device to input an appropriate answer. The types of questions that are sequentially displayed on the monitor screen are: When did this start? This is to determine whether there has been lifelong dysfunction or the dysfunction has been acquired. When does this problem happen? This is to determine situational dysfunction versus a generalized form of dysfunction. Is this caused by a medical condition or medical procedure? Do you think this is caused by medication, alcohol, or drugs? (See page 4 of the Appendix for typical answers to each of these questions.)

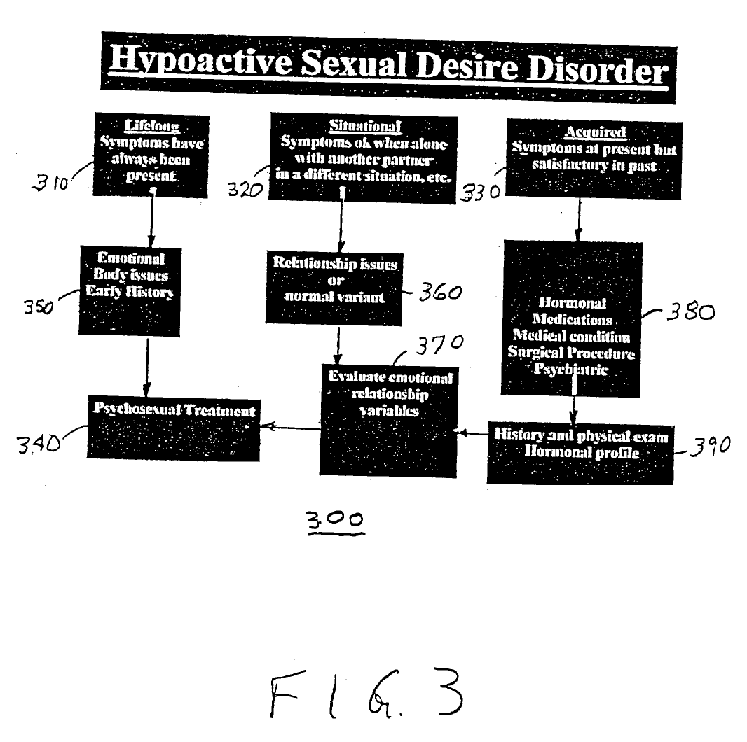

Referring to FIG. 3, algorithmic paths 300 are shown for the three responses of lifelong (box 310), situational (box 320), or acquired (box 330) hypoactive desire disorder. All three responses lead to a recommendation for psychosexual treatment (box 340). The user having lifelong dysfunction has emotional issues, physical (body) issues, or early childhood history of sexual abuse (box 350) that leads the user to psychosexual treatment (box 340). These causes for dysfunction are discussed below. The user with situational dysfunction has either relationship issues or is a normal variant of the entire population (box 360) that must be further examined and evaluated (box 370) before psychosexual treatment (340) is recommended by the FSDA instrument. Finally, the user with acquired sexual dysfunction has hormonal, medications, medical condition, surgical procedure, or psychiatric issues (box 380) that first must be examined from a medical history, physical examination, or hormonal profile (box 390) standpoint and then the user must be further examined and evaluated (box 370) before psychosexual treatment (box 340) is recommended.

After the user has completely answered these series of questions from the first question, the answers are stored in database 60. Preferably, the user continues to answer the second, third, fourth, and fifth Phase one questions until Phase One is complete.

The second Phase One question is broken down into two parts and relates to physical and subjective sexual arousal disorder when a woman does not feel a sexual response in her body or begins to respond but cannot maintain such a response.

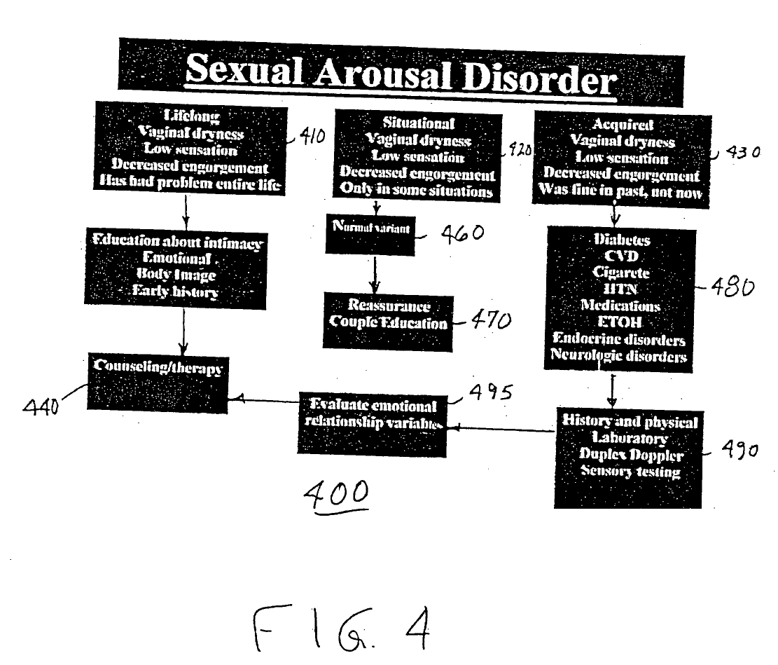

Referring to FIG. 4, algorithmic paths 400 are shown for the same three types of responses discussed above in connection with FIG. 3, lifelong (box 410), situational (box 420), or acquired (box 430) sexual arousal disorder. Lifelong and acquired dysfunction responses lead to a recommendation for psychosexual treatment (box 440). The user having lifelong dysfunction typically has intimacy issues, emotional issues, physical (body) issues, or early childhood history of sexual abuse (box 450) that leads the user to psychosexual treatment (box 440). The user with situational dysfunction is more often considered to be a normal variant of the entire population (box 460). Such a user should be reassured and she and her partner should be given education and guidance (box 470). Finally, the user suffering acquired dysfunction, which may be caused by diabetes, cardiovascular disease (CVD), cigarette smoking, hypertension (HTN), medication problems, ETOH (ethyl alcohol) problems, endocrine disorders, or neurological disorders (box 480), must first be examined. This includes a medical history, a physical examination, medical testing including blood work, an ultrasonographic study, and sensory testing (box 490). The causes must then explored to evaluate the need for further psychological exploration or psychological treatment. The user must be further examined and evaluated (box 495) before psychosexual treatment (box 440) is recommended by the FSDA instrument.

A typical physical sexual arousal disorder question is: Is it ever difficult to achieve or maintain lubrication (wetness) throughout sexual activity of any kind? If the user answers “often” to this question, the interim report will conclude that it is apparent to the FSDA instrument the user suffers a significant physical arousal difficulty, due to inability to achieve and maintain wetness throughout sexual activity. If the user answers “always” to this question, the interim report will conclude that the FSDA instrument has evaluated that the user suffers a great deal of physical arousal difficulty, due to inability to achieve and maintain wetness during sexual activity.

A subjective sexual arousal disorder question is: Do you ever have difficulty getting aroused or feeling emotionally stimulated during sexual activity of any kind? If the answer is “often” to this question, the interim report will conclude that the FSDA instrument has determined that the user suffers a significant difficulty with subjective arousal, due to inability to become aroused (excited) and/or emotionally stimulated during sexual activity. If answer is “always” to this question, the interim report will conclude that the FSDA instrument has determined user suffers a great deal of subjective arousal difficulty, due to inability to become aroused and/or emotionally stimulated during sexual activity.

The third Phase One question relates to orgasmic disorder when a woman has difficulty with orgasm. A typical female orgasmic disorder question is: Is it ever difficult or impossible for you to reach orgasm with sexual activity of any kind?

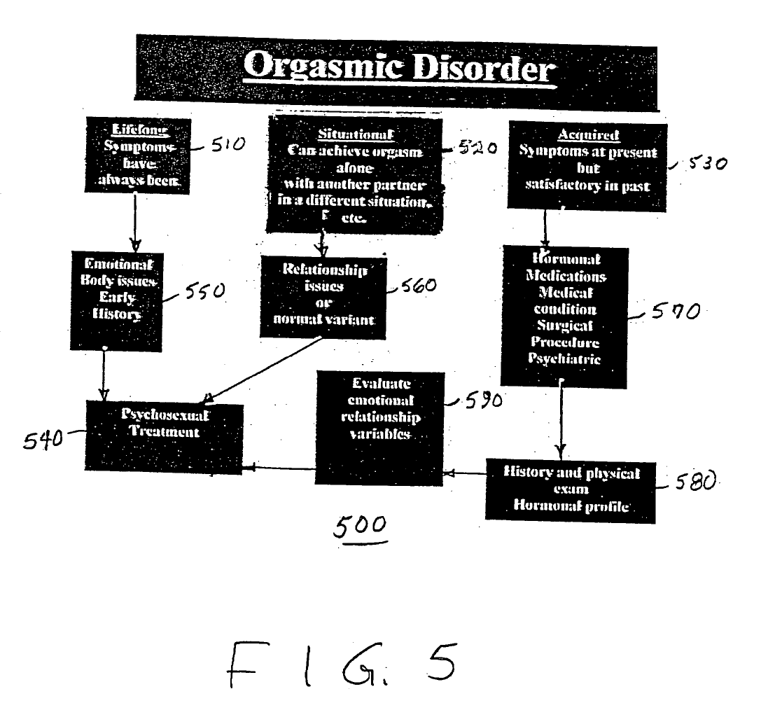

Referring to FIG. 5, algorithmic paths 500 are shown for the same three types of responses discussed above in connection with FIG. 3, lifelong (box 510), situational (box 520) and acquired (box 530) orgasmic disorder. All three disorder responses can lead to a recommendation for psychosexual treatment (box 540). The user having lifelong disorder often has emotional issues, physical (body) issues, or early childhood history of sexual abuse (box 550) that leads the user to psychosexual treatment (box 540). The user with situational disorder usually has either relationship issues or is considered to be a normal variant of the entire population (box 560) that should be given psychosexual treatment (box 540). Finally, the user with acquired disorderstypically has hormonal, medication side effects, medical condition, surgical procedure, or psychiatric issues (box 570) that first must be examined from a medical history, physical examination, or hormonal profile (box 580) standpoint and then the user must be further examined and evaluated (box 590) before psychosexual treatment (540) is recommended the FSDA instrument.

The fourth Phase One question relates to sexual aversion disorder resulting aversion to all or nearly all genital contact with a partner. A typical female sexual aversion disorder question is: Do you ever try to avoid all or nearly all genital sexual contact with a partner?

Refer to FIG. 3 showing the algorithmic path for hypoactive sexual desire disorder to show the path that is also followed with sexual aversion disorder.

The fifth Phase One question relates to sexual pain disorders, which are characterized by pain during or immediately following intercourse. This question is broken down into questions relating to dypareunia, vagnismus, and noncoital pain disorder. A typical dypareunia question is: Do you ever have pain in (or around) your genitals before, during, or after sexual intercourse? A typical vagnismus question is: Do you ever have vaginal spasms that (would) make sexual intercourse difficult or impossible? A typical noncoital pain disorder question is: Do you ever have genital pain during sexual activity that does not involve sexual intercourse?

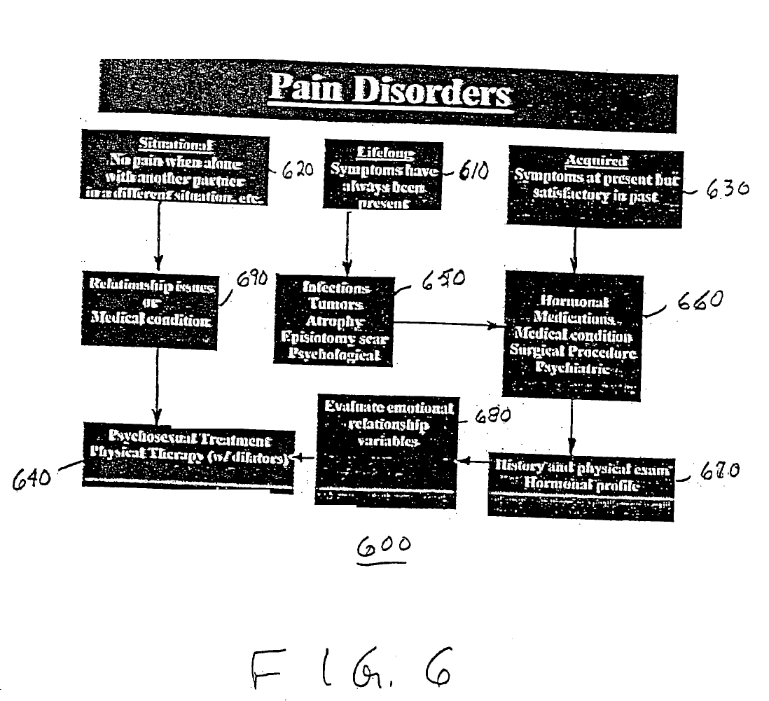

Referring to FIG. 6, algorithmic paths 600 are shown for the same three types of responses discussed above in connection with FIG. 3, lifelong (box 610), situational (box 620) and acquired (box 630) pain disorders. All three responses can lead to a recommendation for either psychosexual treatment or physical therapy, often with the use of dilators (box 640). The user having lifelong pain disorders (box 610) often has infections, tumors, atrophy, episiotomy scar, or psychological disorders (box 650), or has hormonal, medications, medical condition, surgical procedure, or psychiatric issues (box 660). If the user with lifelong pain disorders (box 610) has any of the foregoing disorders or issues, she first must be examined from a medical history, physical examination, and hormonal profile (box 670) standpoint and then the user must be further examined and evaluated (box 680) before psychosexual treatment (box 640) is recommended by the FSDA instrument. The user with situational pain disorders (620) typically has either relationship issues or some type of medical condition (box 690) that leads to a recommendation for either psychosexual treatment or physical therapy (box 640). Finally, the user with acquired pain disorders (box 630) may have hormonal, medications, medical condition, surgical procedure, or psychiatric issues (box 660) that first must be examined from a medical history, physical examination, and hormonal profile (box 670) standpoint and then the user must be further examined and evaluated (box 680) before psychosexual treatment or physical therapy (640) is recommended.

In each of the questions that the user responds with either an “often” or an “always,” the respective interim report will be displayed that indicates the user is probably suffering from either a significant or a great deal of difficulty with respect to that dysfunction of the particular question as discussed above.

In the event the user answers “never” to all five of the questions of Phase One including the subparts concerning whether the user has any of sexual function problems discussed above, the conclusion is that the user has indicated no problems that suggest hypoactive desire, physical sexual arousal difficulty, subjective sexual arousal problems, orgasmic disorder, sexual aversion, dyspareunia, vaginismus, and noncoital pain. The analysis is complete and no further questions will be displayed on the monitor screen and Phase One has ended. The user will receive a printout of a very brief report containing the questions, the answers and the conclusion.

In the event the user answers “occasionally” to all of the questions of Phase One, the user will receive an interim report with the following conclusion: While it is evident that the user has occasional difficulty with hypoactive desire, physical sexual arousal difficulty, subjective sexual arousal problems, orgasmic disorder, sexual aversion, dyspareunia, vaginismus, and noncoital pain, this did not rise to clinical levels of concern or a need for psychological or psychosexual therapy. If the user answers “often” or “always” to any of the questions of Phase One, the user will receive a report containing a summary of the interim reports discussed above based on each individual question.

After the five questions of Phase One have been asked and answered, the database file in database 60 established for each user will be evaluated to determine if any responses of “occasionally,” “often,” or “always” were given to certain of the five questions. They are the first question, the second question, the second part of third question, and the first and third parts of the fifth question. The questions respectively relate to low sexual desire, difficulty in becoming sexually aroused, difficulty achieving orgasm, pain during intercourse, and sexual pain other than during intercourse. If this is the case, a series of questions relating to whether or not any of these symptoms of dysfunction were first noticed after a pregnancy has ended. An example of the question displayed to the user is: Did you first notice low sexual desire after a pregnancy has ended. The user will be asked to respond to one of the following six answers: (1) “No;: (2) “Yes, after a normal vaginal delivery;” (3) “Yes, after a very difficult vaginal delivery;” (4) “Yes, after a Caesarean Section (CS);” (5) “Yes, after having an abortion; or (6) Yes, after a miscarriage.”

An answer of (2), (3) or (4), if the user has low sexual desire, results in a preliminary diagnosis of a potential drop in hormonal levels. Blood tests for testosterone level could assess this. An answer of (3), (4) or (5), if dyspareunia or noncoital pain is present, results in a preliminary diagnosis of pelvic infection or perforated uterus that should be urologically evaluated. An answer of (3), (4) or (5), if orgasmic disorder is present, results in a preliminary diagnosis of potential vaginal or vulvar damage that that should be urologically evaluated. An answer (5) or (6), if the user has low sexual desire, results in a preliminary diagnosis that psychological factors may be playing a role and counseling is advised. (See page 12 of the Appendix for further details of recommendations that are made based on the responses to the foregoing questions.)

An answer of (1) in the negative indicates the user has no sexual function problems related to post-pregnancy. If the user provides an answer of (2) through (5), the user will then be asked: Have any of the following recently troubled you? (a) severe depression after giving birth; (b) the complete attention of the newborn child injured the primary relationship to the father; (c) none of the above has been true.

If the answer is (a) above, the preliminary diagnosis is that such depression following a birth suggests postpartum depression may be involved in sexual problems. It there are symptoms such as crying, sadness, loss of appetite, lethargy, or even thoughts of suicide or hurting the baby, professional psychotherapy is needed to address this. Hormonal drops after giving birth can induce depression, and medical evaluation is advised, as well.

If the answer is (b) of paragraph 52 above, the preliminary diagnosis suggests that the attention demands required by a new baby/child may take the mother's attention away from the earlier, exclusive relationship with the father. This will affect sexual interest, desire, and very likely result in sexual problems. Counseling of a professional to explore this is advised.

At the conclusion of Phase One, the areas relevant to women's sexual dysfunction have been examined. Except for those that have answered “never” to all of the Phase One questions, the user continues with the assessment with additional questions of Phase Two and Three. The questions of Phase Two and Three are of increasing depth depending on detection of problems at the initial screening level during Phase One. It is only after the user has answered the questions of Phase Two and Three, can a determination be made whether the user should seek pharmacological treatment, e.g., Viagra and similar drugs that have significant promise for treating physiologically based sexual function complaints. (See pages 4-12 of the Appendix for the types of the specific questions, typical responses and recommendations for Phase One of the FSDA method.)

PHASE TWO: In step 100 shown on FIG. 2, a number of questions are displayed on the monitor screen relating to either psychological red flags or casual factors for FSD. These red flags fall in one or more of the following categories: mood disorders, anxiety disorders, panic disorders, eating disorders, and gender orientation based on the Diagnostic Statistical Manual of the American Psychological Association (DSM-IV) criteria in accordance with the American Psychiatric Association, as well as self-dissatisfaction, history of sexual abuse, quality of sexual relationships, and sexual orientation. In step 110, the answers to the Phase Two questions and the other responses sent via system 40 to the user's file in database 60 will determine whether the user suffers from any of these psychological causal factors or red flags for FSD.

The first red flag of Phase Two relates to the user's mood. A typical question is: Have you ever had a time where you felt very low or very depressed? The user is provided with the following four possible answers: (1) “No, never for more than a week;” (2) “Yes, when I suffered a great loss;” (3) “Yes, due to a medical problem;” or (4) “Yes, for more than a week and not due to a loss or medical problem.”

An answer set forth under (1) of paragraph 0057 above will result in a conclusion that no significant experience with depression was reported without any evidence of current or past depression by the answer to the mood question of Phase Two.

An answer set forth under (2) of paragraph 0057 above leads to a question whether the symptoms of depression due to bereavement/loss occurred more or less than six months ago. A conclusion is reached that clinical depression would not be evident if the symptoms have not occurred within six months from the FSDA. More recent symptoms would result in a recommendation that professional evaluation of recent depression due to bereavement/loss should be evaluated for possible effect on sexual function. (See page 18 of the Appendix for additional information of this red flag.).

An answer set forth under (3) of paragraph 0057 above again leads to the same type of question as with an answer set forth under (2), whether the medical problem was more or less than six months ago. The same conclusions can be drawn as set forth above.

An answer set forth under (4) of paragraph 0057 above that the depression has gone on for more than a week and it is not due to a great loss or medical problem leads to a series of questions such as: Did you feel very depressed (i.e., sad or empty) most of the day? Did you lose interest in (or stop enjoying) nearly all activities most of the day? How long ago was your last episode of depression (i.e., the above problems)? In an algorithmic path of the type shown in FIGS. 3-6, depending on the user's answers, another series of questions is asked such as: Did you get any help for your last episode of depression? Did you lose a lot of weight (5% or more) without trying to, i.e. when not dieting? Did you gain a lot of weight (5% or more) without trying to? Did you have serious sleeping problems (e.g., sleeping too much or not at all)? Did you feel (or did others tell you that you looked) very restless? Did you feel (or did others find you) very lethargic and slowed down? Did you feel like you had no energy or very fatigued? Did you feel worthless? Did you feel extremely guilty? Did you have trouble thinking or concentrating, e.g., making simple decisions? Did you have recurrent thoughts? (See pages 19-20 of the Appendix for typical answers and the algorithmic path followed by the FSDA instrument depending on the answers as well as the preliminary diagnosis for such answers.).

In the worst-case scenario, a user may either have major depressive episodes or have a major depressive disorder. (See pages 19-20 of the Appendix.) In the former case of a major depressive episode (MDE), a report on the user is prepared with list of severe and mild symptoms suffered by the user and when they occurred.

A typical list of mild symptoms of a mood disorder that have lasted at least 3-7 days include: depressed mood, loss of interest in daily activities, sleeping problems, restlessness lethargy/slowing, fatigue feelings of worthlessness, feeling guilty, and inability to concentrate/indecisiveness.

A typical list of serious or severe symptoms of a mood disorder that have lasted for more than a week include: depressed mood, loss of interest in daily activities, weight loss, weight gain, sleeping problems, restlessness, lethargy/slowing, fatigue feelings of worthlessness, feeling guilty inability to concentrate/indecisiveness, recurrent thoughts of death/dying. To deal with this depression that raises to a clinical major mood disorder or depression a report is sent to the user with the recommendation to seek help.

A similar preliminary mood disorder diagnosis is given for both serious and mild symptoms. Symptoms that have occurred on the day of the FSDA to within the past few weeks from the assessment or continue to be present could be expected to cause sexual dysfunction. To summarize, preliminary mood disorder diagnosis relative to the date of the FSDA are as follows: (1) symptoms that occurred in the past month or occurred very recently, may still be causing some sexual dysfunction; (2) symptoms that occurred 2 months to a year ago or occurred in recent months, may still be causing sexual dysfunction; (3) symptoms that occurred within past 2 years or occurred within the past two years, may still cause some degree of sexual dysfunction; or (4) symptoms that occurred within 3 or more years ago or occurred more than two years ago, are unlikely to be affecting current sexual dysfunction.

In the case of a major depressive disorder, user counseling should have a positive effect on sexual dysfunction if: (1) the user is in counseling that addresses the effects of sexual dysfunction; (2) the user was in counseling and reinitiated counseling that addresses the effects of sexual dysfunction; or (3) the user was never in counseling but agrees to begin counseling that addresses the effects of sexual dysfunction. (See pages 19-20 of the Appendix for details of recommendations for mood disorders.)

The second red flag of Phase Two relates to the user's anxiety. A typical question is: Have you ever felt panic and/or very anxious, agitated, or constantly worried for more than a week? The same four Phase Two answers set forth under (1), (2), (3) or (4) above under paragraph 0057 that were the possible answers under the first red flag question relating to mood are used with anxiety and the same preliminary diagnosis for such answers. Similarly the answer in the affirmative to the question (4) results in the same type of algorithmic paths as under mood. (See pages 21-22 of the Appendix for typical answers and the algorithmic path followed by the FSDA instrument depending on the answers as well as the preliminary diagnosis for such answers.)

The third red flag of Phase Two relates to the users possible panic attacks. A typical question is: Have you ever had a panic attack? A panic attack is defined as an intense fear or anxiety that causes both physical and emotional distress. Answer of “yes” to this third red flag question should be given by the user only if at the attack's peak the user felt so much fear/anxiety she almost could not stand it. The user is provided with the following five possible third red flag answers: (1) “No, not really;” (2) “Yes, and the attack built to a peak over four hours or more;” (3) “Yes, and the attack built to a peak within one to three hours;” (4) “Yes, and the attack built to a peak within 11 minutes to an hour;” or (5) “Yes, and the attack built to a peak within ten minutes or less.”

A red flag answer set forth under (1) above under paragraph 0057 will result in a conclusion that no significant experience with panic-like episodes was reported with no evidence of current or past panic like episodes by the answer to the panic like episodes question of Phase Two.

In all of the red flag answers set forth under (2) through (5) above under paragraph 0057, the following questions are asked: How often do these attacks occur? How long ago was your last episode of anxiety/worry? Depending on the answers, one or another algorithmic path is followed to lead to a proper diagnosis. One path is for those that answered with (2) through (4) and a slightly different path is followed for those that answered with answer (5). (See pages 23-24 of the Appendix for examples of other algorithmic paths that the user can follow.)

The fourth red flag of Phase Two relates to the user's eating habits. A number of questions are asked to determine if the user is suffering from an eating disorder that affect sexual function such as anorexia nervosa, anorectic eating disorder features, bulimic eating disorder features, bulimia nervosa, and eating disorder with body image distortion. (See pages 25-27 of the Appendix for examples of the type of questions that are asked and typical answers that are given to this red flag area.)

The fifth red flag of Phase Two relates to the user's possible self-dissatisfaction. Questions are asked to determine how the user feels about herself. The following areas are covered during the question and answer phase of this fifth red flag: self image, physical attractiveness, genital appearance, social skills/personality, weight and size, feeling healthy, expectations for the future, and economic status. This part of the assessment requires a mathematical determination based on rating each of the above areas of self-satisfactory or dissatisfaction with oneself. The ratings range from a score of 0 to 5 for the following answers: none, some, moderate, fairly high, high, and very high. An example is given on page 29 of the Appendix showing that if a women has a score of 1 or greater in physical appearance, weight/size and social skills/personality and a score of 2 or higher for genital appearance, counseling is recommended. These scores on a user's anxiety and avoidance of sexual situations as well as a reduction of her confidence are too high for her to ask for what she needs (sexually) in a relationship. (See pages 28-31 of the Appendix for the type of questions and typical answers to determine how the user feels about herself.)

The sixth red flag of Phase Two relates to the user's possible history of sexual abuse. A typical question is: Tell us if you have had any experiences with sexual abuse; i.e., Did anyone ever force any type of sexual experience on you; such as touching you in a way that made you feel very uncomfortable? The first question is to determine whether there are any incidences of sexual abuse. The next questions relate to whether there has been any support from the people the woman has told about any such incidences. Finally, the questions are designed to elicit whether the user has perceived a degree of resolution. (See pages 61-63 of the Appendix for the type of questions and typical responses on the user's sexual abuse history.)

The seventh red flag of Phase Two relates to the quality of the user's sexual relationships. The first questions are to determine if the user has a sexual partner or any problems interfering with starting a relationship. The next series of questions relate to the users current sexual relationship, its duration, the user's attitude toward her partner, perception of partner's attitude, understanding the user's emotional needs, understanding her sexual needs, feeling safe in the relationship, liking each other's personality, and finding each other physically attractive. (See pages 64-66 of the Appendix for the type of questions and typical responses on the quality of the user's sexual relationships.).

The eighth red flag of Phase Two relates to the user's sexual orientation/gender orientation. Gender orientation problems include gender identity disorder (according to DSM-IV criteria) and the presence of gender identity conflict and are associated with a user being uncomfortable with her physical gender; i.e., her body's sexual characteristics. On the other hand, a sexual orientation problem is associated with the presence of conflict with sexual orientation. The importance of questions in this area is to evaluate the potential impact of conflicts, if they are present, on the user's sexual relationships. (See pages 67-68 of the Appendix for the type of questions and typical responses on the quality of the user's sexual orientation/gender orientation.)

PHASE THREE: In step 120 shown on FIG. 2, a plurality of questions is displayed relating to medical factors affecting sexual function. In step 130, the answers to the question raised during Phase Three questions and the other responses are sent via system 40 to the user's file in database 60 and will determine whether the user has any medical factors that impact on sexual function. The questions relate to the user's use of tobacco and alcohol, the user's medical history, medications including any selective serotonin re-uptake inhibitors (SSRI) antidepressants that the user is currently taking, the user's menstrual status, and pain disorder in general or associated with a medical condition or somatization disorder (as per DSM-IV criteria).

It is not critical to the FSDA method that all of the Phase Two questions and responses be given by the user before the user's screen displays Phase Three questions for a response. In other words, the sequential steps shown in FIG. 2 are not necessarily followed when it comes to Phases Two and Three. The only steps that are followed in the preferred embodiment of the FSDA method, before the user's screen displays any of the Phase Two and Three questions, are steps 50, 70, 80 and 90 of FIG. 2 relating to (a) registration, (b) gathering of demographic information, (c) receiving the sexual dysfunctional questions, and (d) providing the answers to the sexual dysfunctional questions. After the steps (a)-(d) have been taken, all users that have not responded “no” or “never” to all the sexual dysfunctional questions will have questions from Phase Two and Three displayed. For example, a few Phase Two questions may be interchanged with a few Phase Three questions. (See pages 18 through 68 of the Appendix for one example of the interchanging of questions from Phases Two and Three.)

An attempt is made in the FSDA method, after the Phase One questions have been answered, to wait until the latter stages of the assessment to ask questions that are of less importance either from the standpoint of the greatest majority of respondents or because answers to certain medical factors are more important in assessing FSD than some of the Phase Two questions. For example, the Phase Two questions relating to sexual orientation/gender orientation are usually of less importance and are near the last set of the questions in the FSDA. The numbering below of the Phase Three questions from one through six below is arbitrary and not critical to the FSDA.

Questions one and two of Phase Three are: “Do you use tobacco?” and “Do you use alcohol?” They are taken in sequential order. They both can be respectively answered with (1) “No never (other than trying it);” (2) “Not now, but I used to;” or (3) “Yes, I use tobacco at this time;” or “I currently drink alcohol.” For example, the user receives a report with the recommendation that fairly heavy as well as heavy cigarette smoking is likely to interfere with sexual function as well as many other aspects of health and that smoking cessation is advised. (See pages 32-34 of the Appendix for typical answers for tobacco use with page 34 for recommendations depending on the frequency of current tobacco use.)

The CAGE questionnaire for alcohol abuse is used to determine whether the user not only suffers from alcohol abuse, but that such use impairs sexual responsiveness and interest as well as interfering with good judgment regarding sex and safe precautions. Have you ever felt you should Cut down your alcohol use? Has anyone ever Annoyed you by criticizing, or complaining, about your use of alcohol? Have you ever felt Guilty or upset about using alcohol? Have you ever had a drink first thing in the morning to steady your nerves (i.e., an Eye-opener) or to get rid of a hangover? The combination of the initial letter of Cut, Annoyed, Guilty, and Eye-opener from the four questions is the origin of the CAGE acronym. (See pages 35-37 of the Appendix for typical answers for alcohol use.)

Question three of Phase Three is directed to the user's medical history and includes a checklist of current medical problems or conditions, a medical history of past problems and conditions that may affect sexual function. (See pages 38-42 of the Appendix for a detailed checklist of medical problems that the user could be having.)

Current medical issues of this checklist that may have such an effect include: coronary heart disease (atherosclerosis), high blood pressure, high cholesterol, insulin dependent diabetes mellitus (IDDM or Type I) that is not gestational, non-insulin dependent diabetes mellitus (NIDDM or Type II) that is not gestational, and hypothyroidism.

Previous operations/procedures on the checklist that can affect sexual function include: total hysterectomy (removal of the uterus and cervix), partial hysterectomy (removal of the uterus), uterine embolization, unilateral oophorectomy (removal of one ovary), bilateral oophorectomy (removal of both ovaries), myomectomy (removal of uterine fibroids), removal of a uterine cyst, dilation and curettage (D&C), rectal surgery, radical mastectomy, modified radical mastectomy, total mastectomy, lumpectomy, reconstructive breast surgery.

The following infections and inflammations on the checklist that can result in orgasmic problems and dyspareunia include: candida (yeast infection), trichomoniasis (Trich), bacterial vaginosis (gardnerella or hemophilus), urinary tract infection (UTI, cystitis or other type), toxic shock syndrome (TSS), vaginitis, vulvitis, interstitial cyctitis (bladder disease), and vulvodynia.

Dyspareunia or non-coital pain can be caused by pelvic inflammatory disease (PID) are on the checklist and they can include: endometritis (infection of the lining of the uterous), salpingitis (infection of the fallopian tubes), oophoritis (infection of the ovaries), and PID that combines two or more above or if the user is not sure of the type.

The checklist includes spinal cord injury, bicycle riding more than once per week using a standard bicycle seat, pelvic fracture, traumatic (very difficult) childbirth, and female circumcision. A report will be sent to the user that physical arousal, dyspareunia, non-coital pain, or orgasmic problems can result from such injuries and trauma.

A report will also be sent to a user that hypoactive desire, non-coital pain, dyspareunia are some of the FSD problems associated with sexually transmitted diseases (STD). The checklist for STD includes: trichomoniasis, human papillomavirus, Chlamydia, herpes, gonorrhea, hepatitis B, syphilis, and HIV. In addition, the report will state that STD is probably a contributing factor to genital pain discomfort during non-coital and/or coital sexual relations, as STD can have many symptoms, presenting at different times and in different body locations. The report will recommend that if the user is not already in treatment, medical advice should be sought immediately for STD.

The report will state that there is evidence of subjective sexual arousal difficulty that could be related to self perception of disfigurement (poor body image) due to female circumcision and breast surgery lowering your self confidence or to autoimmune disorder causing fatigue, which includes: chronic fatigue syndrome, fibromyalgia, scleroderma, Sjogren's syndrome, systemic lupus erythematosus.

Finally, the checklist includes at least the following types of cancer that are known to affect sexual function: breast cancer, ovarian cancer, uterine cancer, and other cancers such as pelvic cancer.

Question four of Phase Three is directed to the medications that the user is currently taking. The following seven categories of drugs can affect sexual function: (1) antihypertensives such as chlorthalidone (Hygroton, Thalidone), guanadrel (Hylorel), guanesthidine (Ismelin), methyldopa (Aldomet), reserpine, spironolactone (Aldactone); (2) antidepressants such as clomipramine (Anafranil), imipramine (Tofranil, Janimine), phenelzine (Nardil), and SSRI (Effexor, Luvox, Paxil, Prozac, Zoloft); (3) other psychiatric medications including chlorpromazine (Thorazine), clomipramine (Anafranil), fluphenazine (Prolixin, Permitil), lithium (Eskalith, Lithonate), pimozide (Orap) and thioridazine (Mellari); (4) illicit or overused/abused substances such alcohol (three or more drinks per day as referred to above under use of alcohol), amphetamines (used regularly), amyl nitrite (poppers) barbiturates (regular use), cocaine, diazepam (Valium, Zanax, and similar tranquilizers if used regularly); (5) methylenedioxy-methamphetamine (MDMA) including marijuana (used regularly), methaqualone (qualudes), morphine (MS Contine, Oxycontin, Roxanol used regularly), tobacco (smoking two or more packs per day as referred to above); (6) other miscellaneous drugs affecting sexual desire include acetazolamide (Diamox Ak-Sol), carbarnazepine (Tegretol, Atretol, and similar drugs), clofibrate (Atromid S), danazol (Danocrine), dicyclomine (B enryl, Di Spaz, and the like), digoxin (Lanoxin), esthinyl, estradiol (Estanyl), hydrochlorthiazide (Esidrix, HyrodDiURIL, Oretix, and the like), ketoconazole (Nizoral), levodopa (Larodopa, Dopar), methadone (Dolophine), methazolamide (Neptazane), norethindrone (Norlutin), phenobarbitol phentoin (Dilantin), primidone (Mysoline), and thiabendazole (Mintezol) ; and (7) over-the-counter drugs such as niacin (Nicolar, Niacor, Nicobid). (See pages 43-44 of the Appendix for additional information on current medication usage and pages 54-56 of the Appendix for additional questions regarding SSRI depressants usage.)

The following medications from the checklist for question four of Phase Three, for example, can lower sexual desire in a user: antihypertensives such as chlorthaliclone, guanadrel. guanesthidine (if taken very frequently), methyldopa, reserpine, spironolactone, the antidepressant such as clomipramine, the psychiatric medications of fluphenazine (if taken very frequently), danazol, esthinyl estradiol, ketoconazole, methadone (if taken very frequently), the over-the-counter drug niacin, and alcohol, barbiturates, diazepam, marijuana, methaqualone, morphine.

The following medications from the checklist, for example, can increase sexual desire include the anti depressant imipramine, the psychiatric medications, chlorpromazine, clomipramine (if taken very frequently), fluphenazine (if taken very frequently), lithium, pimozide, thioridazine (very frequently) acetazolamide, carbamazepine, clofibrate, clanazol (very frequently), dicyclomine, digoxin, hydrochlorthiazide (very frequently). ketoconazole (if taken very frequently), levoclopa, methadone (if taken very frequently), methazolamide, norethindrone, phenobarbitol, phentoin, primidone, thiabendazole, the over-the-counter drug, niacin, alcohol, amphetamines, amyl nitrite, barbiturates, cocaine, MDMA (if taken very frequently), methaqualone, morphine, and tobacco.

Question five of Phase Three relates to menstrual status and whether the user is in menopause or suffers from premenstrual syndrome (PMS). Depending on the user's answers a report will be provided to the effect that moderate, severe and very severe menopausal difficulties or PMS discomfort would probably erode a woman's self confidence and reduce her sexual responsiveness and motivation. (See pages 45-48 of the Appendix for other examples of the type of questions and responses in this category.)

Question six of Phase Three relates to pain problems the user may be facing that affect sexual function. An example of question six is: Have you had any serious pain or discomfort in the past month? The user is then asked to check any serious pain that interfered with the user's life over the past month: wrist or hands pains, headache, feet, bloating or gas, back, chest, belly or stomach distress, arms, neck, get sick from several foods, leg, knee, jaw or face, ankle, joint, pain while urinating, rectal, hip, shoulders, or none of the above. The user is then asked to check off any severe digestion problems during the past month such as: nausea or upset stomach, constipation, vomiting (when not pregnant), diarrhea, bloating, gets ill from different foods, or none of the above. The user is then asked whether any of these pain disorders are associated with a medical condition, stress, emotional upset or emotional conflict and the duration for these problems. An analysis is made and a determination is made whether the user is suffering from a pain disorder or a somatization disorder as defined by the DSM-IV. A very brief definition of a somatization disorder is that it is a chronic condition in which there are numerous physical complaints that have lasted for a number of years and have resulted in substantial impairment, which are caused by psychological problems.

If a somatization disorder is diagnosed, questions are asked to find out the age of onset, its duration, and whether the disorder is due to a medical condition/drug side effect. The next step is to determine whether the user is suffering from an undifferentiated somatization disorder or another type such as conversion disorder, physical symptoms caused by psychological conflict, unconsciously converted to resemble those of a neurological disorder. In that case, the user is further questioned to determine if the physical symptoms are with motor symptoms, sensory symptoms, seizures/convulsions, or a mixed presentation. (The details of how these responses are analyzed and scored to enable the FSDA instrument to come up with such a determination are found on pages 49-53 of the Appendix.)

In a preferred embodiment of the method of the present invention, an additional step is added to the steps of Phases One, Two and Three. During this step, questions are displayed on the user's screen relating to the quality of the user's orgasm. The responses given to these questions are sent to the user's file in database 60. A report is sent to the user's computer system 20 containing a chart showing the user's satisfaction level with the orgasm quality. The user's satisfaction level includes satisfaction with the orgasm intensity, how easily orgasm is achieved, satisfaction with quality of orgasm, fulfillment after orgasm, and orgasm enjoyment. These responses from the orgasm quality questions are very useful when taken in combination with the responses to the Phase One, Two and Three questions. During step 140, the total of these responses are evaluated to determine whether the user presents herself as a suitable candidate for medical treatment to enhance sexual function, such as a prescription for a Viagra-type of product. For example, a user is determined to be a suitable candidate for such products if there are no psychological or relationship causes for FSD and the user has no more than a fair satisfaction level on the question of orgasm enjoyment based on this evaluation of the quality of the user's orgasms. (See pages 13-17 of the Appendix for details of the type of questions asked and the type of responses from the user that is gathered to determine the user's quality of orgasms.)

Another step of the preferred embodiment of the present invention is to display on the user's screen, questions on the user's self-improvement and general interests. The questions relating to self-improvement fall into the following categories: self-confidence, stress management, money management, personal organization, and self-image. The questions relating to general interest fall into the following categories: nutrition, habit control, social or family relationship areas, exercise, weight loss, travel, career building, and personal growth. The user's responses are sent to the user's database file in database 60 for evaluation. While this step is not necessary to complete the FSDA of the user, the information is useful in providing a more complete analysis of the user. Upon completion of this step, a report is sent to the user's computer system 20 for printout if desired. (See pages 56-60 of the Appendix of the checklist combining self-improvement and general interests and the type of reports that the user will receive.)

The FSDA method and systems described above is intended be used to rule out FSD and it's causal factors by physicians, psychologists and psychotherapists, medical researchers, pharmaceutical researchers and women who would like to assess themselves and decide whether to seek help and determine what kind of help they may need. The FSDA instrument can be used on patients, research subjects and the general public to arrive at a preliminary diagnosis of whether any of these users is a suitable candidate for medical therapy or psychological therapy to enhance sexual fumction or both of these therapies.

While various embodiments of the method and system of the present invention have been described, these embodiments are not intended to limit the scope of the present invention, which is set forth in the appending claims. Various modifications of the above described embodiment can be made by those skilled in the art after reading the specification of the subject application. These modifications are within the scope and true spirit of the present invention.

What is claimed is:

1. A method of performing an automated sexual function assessment of a female user comprising the following steps:
(a) displaying a plurality of female sexual dysfunction (FSD) questions to the user during a first phase of the assessment;
(b) sending to a central database the user responses to the plurality of FSD questions;
(c) displaying a plurality of questions to determine if the user has any psychological causal factors for female sexual dysfunction in a second phase of the assessment if the responses to the questions of step (a) are other than never;
(d) sending to the central database the user responses to the plurality of questions displayed to the user in step (c);
(e) displaying a plurality of questions to determine if the user has any medical factors impacting on sexual function in a third phase of the assessment depending on the responses received in step (d) of the second phase;
(f) sending to the central database the user response to the plurality of questions displayed to the user in step (e); and
(g) evaluating the user's responses obtained during the first, second, and third phases to determine whether the user is a suitable candidate for medical therapy or psychological therapy to enhance sexual function or both of these therapies.

2. The method of claim 1, wherein a password is issued to the user in response to the user registering online.

3. The method of claim 1, the user provides user's demographic information to a central database.

4. The method of claim 2, the user sends to the central database one of the following responses to the FSD questions in step (a): never, occasionally, often and always.

5. The method of claim 1, wherein the FSD questions of the first phase to determine a preliminary sexual dysfunction diagnosis are in accordance with the National Consensus Panel of the American Foundation of Urologic Disease.

6. The method of claim 5, wherein the FSD questions relate to an area selected from the groups consisting of hypoactive desire disorder, sexual arousal disorder, orgasmic disorder, sexual aversion disorder, sexual pain disorder, and combinations thereof.

7. The method of claim 1, wherein said psychological causal factors are selected from the group consisting of the following: mood disorders, anxiety disorders, panic disorders, eating disorders, self-dissatisfaction, history of sexual abuse, quality of sexual relationships, sexual orientation, gender orientation, and a combination thereof.

8. The method of claim 1, wherein said medical factor questions are selected from the group consisting of tobacco use, alcohol use, medical history, current medications, menstrual status, pain problems, and mixtures thereof.

9. The method of claim 6, wherein before the user is provided with step (c) or step (e) questions responses all of the following FSD questions must be sent to the central database in step (b): hypoactive desire disorder, sexual arousal disorder, orgasmic disorder, sexual aversion disorder, and sexual pain disorders.

10. The method of claim 7 wherein all of the following psychological questions of step (c) are sent to the central database in step (d): mood disorders, anxiety disorders, panic disorders, eating disorders: self-dissatisfaction, history of sexual abuse, quality of sexual relationships, sexual orientation, and gender orientation.

11. The method of claim 8, wherein all of the following medical factor questions of step (g) are sent to the central database in step (h): medical history, current medications, menstrual status, and pain problems.

12. The method of claim 1, including the additional step of displaying a plurality of questions to determine the quality of the user's orgasms.

13. The method of claim 12, including the additional step of displaying a plurality of questions to determine general and self-improvement interests.

14. The method of claim 6, wherein a user that responses with a “never” to all five of the FSD questions of step (a) the questions of step (c) or step (e) are not displayed.

15. The method of claim 14, wherein after the user's responses to all of the questions raised in the first, second, and third phases have been sent to the central database and the responses have been evaluated, a determination is made whether the user is a suitable candidate for medical or psychological treatment to enhance sexual function or a combination of both treatments.

16. The method of claim 12, wherein after the user's responses to all of the questions raised in the first, second, and third phases and the questions on the quality of the user's orgasms have been sent to the central database and the responses have been evaluated, a determination is made whether the user is a suitable candidate for medical or psychological treatment to enhance sexual function or a combination of both treatments.

17. The method of claim 16, wherein a user is a suitable candidate for medical treatment to enhance sexual function if there are no psychological casual or medical factors for FSD and user has no more than a fair satisfaction level on the question of orgasm enjoyment based on an evaluation of the quality of the user's orgasms.

18. A method of performing an automated sexual function assessment of a female user comprising the following steps:
(a) issuing a password to the user in response to the user registering online;
(b) providing the user's demographic information to a central database;
(c) displaying to the user female sexual dysfunction (FSD) questions relating to the areas of hypoactive desire disorder, sexual arousal disorder, orgasmic disorder, sexual aversion disorder, and sexual pain disorders in a first phase of the assessment;
(d) sending to the central database by the user one of the following responses to the central database: never, occasionally, often and always of the questions displayed in step (c);
(e) displaying a plurality of questions to determine if the user has any psychological causal factors for female sexual dysfunction in a second phase of the assessment if any of the responses received during step (d) are other than never, said psychological causal factors being selected from the group consisting of the following: mood disorders, anxiety disorders, panic disorders, eating disorders, self-dissatisfaction, history of sexual abuse, quality of sexual relationships, sexual orientation, gender orientation, and a combination thereof;
(f) sending to the central database the user responses to the plurality of questions displayed to the user in step (e);
(g) displaying a plurality of questions to determine if the user has any medical factors impacting on sexual function in a third phase of the assessment depending on the responses received during step (f) of phase two; said questions being selected from the group consisting of tobacco use, alcohol use, medical history, current medications, menstrual status, pain problems, and mixtures thereof;
(h) sending to the central database the user response to the plurality of questions displayed to the user in step (g); and
(i) evaluating the user's responses obtained during the first, second, and third phases to determine whether the user is a suitable candidate for medical therapy or psychological therapy to enhance sexual function or both of these therapies.

19. The method of claim 18 wherein all of the following psychological questions of step (c) are sent to the central database in step (d): mood disorders, anxiety disorders, panic disorders, eating disorders: self-dissatisfaction, history of sexual abuse, quality of sexual relationships, sexual orientation, and gender orientation.

20. The method of claim 18, wherein all of the following medical factor questions of step (g) are sent to the central database in step (h): medical history, current medications, menstrual status, and pain problems.

21. A system for performing an automated sexual function assessment of a female user using a link between a user computer system and a central computer system comprising:
a remote communication system for initiating a remote communication from the user computer system with said central computer system;
first phase storage on a hard drive within said central computer system for storing a plurality of female sexual dysfunction (FSD) questions of a first phase of the assessment;
second phase storage on a hard drive within said central computer system for storing a plurality of questions of a second phase to determine if the user has any psychological causal factors for female sexual dysfunction if any of the responses received in the first phase are other than never;
third phase storage on a hard drive within said central computer system for storing a plurality of questions of a third phase to determine if the user has any medical factors impacting on sexual function depending on the responses received in the second phase;
display screen on said user computer system for displaying the plurality of questions received via said remote communication system;
response storage on a hard drive within said central computer for storing the responses to said plurality of questions in a separate user file; and
an instrument programmed into said central computer system for evaluating the responses from the plurality of questions during the first, second, and third phases if any of the responses of the first phase questions are other than never and determining whether the user is a suitable candidate for medical therapy or psychological therapy to enhance sexual function or both of these therapies.

22. The system of claim 21 also includes database storage within said the hard drive of said central computer system for storing password and demographic information received from the user via said communication means.
